(19) United States
(12) Patent Application Publication
(10) Pub. No.: US 2026/0224365 A1
(43) Pub. Date: Aug. 6, 2026

(54) IMPLANTABLE VENOUS STENT HAVING ONE OR MORE VALVES SECURED THEREIN, AND RELATED SYSTEMS AND METHODS

(71) Applicant: Bard Peripheral Vascular, Inc., Franklin Lakes, NJ (US)

(72) Inventors: Bryon Ray Densley, Rochester, MN (US); Amanda Young, Phoenix, AZ (US); Brandon Simmons, Tempe, AZ (US); Christy Graham, Apache Junction, AZ (US); Kyra Temple, Mesa, AZ (US); Zachary Willis, Mesa, AZ (US); Jakob Wells, Scottsdale, AZ (US); Greta Raeann Hull, Phoenix, AZ (US); Vincent Jonathan Beck-Jones, Mesa, AZ (US)

(21) Appl. No.: 19/046,754

(22) Filed: Feb. 6, 2025

(51) Int. Cl. A61F 2/24

(52) U.S. Cl. CPC A61F 2/2475; A61F 2230/0069; A61F 2240/001

(57) ABSTRACT

An example venous stent includes a stent body, a sleeve, and a valve. The stent body is elongated and expandable. The stent body has a lumen extending therethrough and is sized and dimensioned to be implanted into a vein. The sleeve is secured to the stent body and at least partially defines the lumen extending through the stent body. The valve is secured to the stent body and the sleeve within the lumen. The valve is configured to inhibit a first direction of blood flow through the valve in the lumen and allow a second direction of blood flow through the valve in the lumen when the venous stent is implanted into the vein.

### BACKGROUND

Chronic venous insufficiency (CVI) and pelvic congestion syndrome (PCS) are conditions that may result from the improper flow of blood through veins in the body. Healthy valves in veins typically keep blood flowing in the veins and back up to the heart of the individual rather than refluxing in the legs (for example) of the individual. In CVI, however, valves in the legs may be damaged, allowing blood to reflux and pool in the legs of the individual. In PCS, blood flows backwards in the pelvic veins, causing chronic pelvic pain from pelvic veins swelling and twisting.

Conventional venous stents may include a metal mesh tube that is inserted into a vein to treat blockages in the veins characteristic of conditions such as deep vein thrombosis (DVT). Conventional venous stents, however, typically are not adequate to treat conditions such as CVI and PCS.

### SUMMARY

Embodiments disclosed herein are related to implantable venous stents having one or more valves secured therein, and related systems and methods. In an embodiment, a venous stent includes a stent body, a sleeve, and a valve. The stent body is elongated and expandable. The stent body has a lumen extending therethrough and is sized and dimensioned to be implanted into a vein. The sleeve is secured to the stent body and at least partially defines the lumen extending through the stent body. The valve is secured to the stent body and the sleeve within the lumen. The valve is configured to inhibit a first direction of blood flow through the valve in the lumen and allow a second direction of blood flow through the valve in the lumen when the venous stent is implanted into the vein.

In an embodiment, a method of forming a venous stent is disclosed. The method includes disposing a valve material between a first mandrel and a second mandrel. The method also includes positioning a liner over the valve material, the first mandrel, and the second mandrel. The method also includes positioning a stent body over the liner and positioning an outer sleeve over the liner to form an assembly. The method also includes compressing and sintering the assembly such that the liner and the outer sleeve are secured together to form a sleeve secured to the stent body, and the valve material is secured to the sleeve and the stent body. The method also includes removing the first mandrel and the second mandrel to form a lumen within the sleeve and the stent body. The method also includes forming a passage through the valve material to form a valve in the lumen configured to inhibit a first direction of blood flow through the valve in the lumen and allow a second direction of blood flow through the valve in the lumen when the venous stent is implanted into the vein.

In an embodiment, a method of inhibiting venous reflux flow and chronic venous insufficiency is disclosed. The method includes implanting a venous stent into a vein. The venous stent includes a stent body that is elongated and expandable. The stent body has a lumen extending therethrough. The sleeve is secured to the stent body and at least partially defines the lumen extending through the stent body. The valve is secured to the stent body and the sleeve within the lumen. The valve allows a first direction of blood flow through the valve in the lumen and inhibits a second direction of blood flow through the valve in the lumen.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a side view of a venous stent having a valve secured therein, according to an embodiment.

FIG. 1B is a cross-sectional view of the venous stent taken along plane 1B-1B.

FIG. 1C is a front view of the venous stent of FIG. 1A.

FIG. 1D is a side view of a venous stent having a plurality of valves secured therein, according to an embodiment.

FIGS. 2A-2H are side cross-sectional views of the venous stent of FIG. 1A during a method of manufacture.

FIG. 3A is a side view of a venous stent having a valve secured therein, according to an embodiment.

FIG. 3B is a cross-sectional view of the venous stent taken along plane 3B-3B.

FIG. 3C is a front view of the venous stent of FIG. 3A.

FIGS. 4A-4H are side cross-sectional views of the venous stent of FIG. 3A during a method of manufacture.

FIG. 5 is a flow diagram of a method of manufacturing a venous stent having a valve secured therein, according to an embodiment.

FIG. 6 is a cutaway side view of a venous stent having a valve secured therein, according to an embodiment.

FIG. 7A is a cutaway side view of a venous stent having a valve in an open configuration secured therein, according to an embodiment.

FIG. 7B is a cutaway side partial cross-sectional view of the venous stent of FIG. 7B having the valve in closed configuration, according to an embodiment.

FIG. 8 is a cutaway side view of a venous stent having a valve secured therein, according to an embodiment.

### DETAILED DESCRIPTION

Embodiments disclosed herein are related to implantable and covered venous stents having one or more valves secured therein, and related systems and methods. In some embodiments, the venous stents disclosed herein include implantable covered stents that house one or more synthetic valves to reduce reflux of blood within the venous system upon implantation. The venous stents disclosed herein may be used to address pelvic venous insufficiency associated with pelvic congestion syndrome (PCS) and/or reflux in other sites within the body. For example, in a non-limiting implementation and with access to the ovarian vein through standard guidewire and catheter techniques, embodiments of the venous stents disclosed herein may be deployed over a guidewire.

Multiple embodiments of venous stents having one or more valves secured therein are disclosed herein, including a self-expandable venous stent or a balloon-deployed venous stent that may or may not be dilated post-deployment. In these and other embodiments, the synthetic valve of the venous stent is configured to operate similarly to a native valve, e.g., where native pulsatile flow would easily open the valve and, due to the one-way flow design of the valve, the valve closes to any refluxing flow. This configuration of the venous stent having the valve secured therein allows venous blood to travel through the vein (e.g., ovarian vein) and reduce pooling in the vascular system (e.g., pelvic vascular system). In some embodiments, a venous stent having one or more valves secured therein of this disclosure may be implanted over existing native valves (that may or may not be damaged) or, for example, along any other sections of a vein (e.g., the ovarian vein).

Embodiments of the implantable and covered venous stents disclosed herein are sized and dimensioned to be implanted into one of more veins of the venous system. For example, embodiments of the venous stents having one or more valves secured therein according to this disclosure may include the following diameter sizes to accommodate the range of ovarian veins present with PCS such as a diameter of about 1 mm to 8 mm, more particularly a diameter of about 3 mm to about 4 mm for a typically sized ovarian vein, about 5 mm to about 6 mm for nominally dilated ovarian veins, and/or about 7 mm to about 8 mm for grossly dilated ovarian veins.

The length of the venous stents disclosed herein may vary according to different embodiments. For example, an embodiment of the venous stent may include a single valve therein, and the length of the venous stent may be the shortest size needed to accommodate the single valve, such as about 10 mm. Some embodiments of the venous stents disclosed herein may include multiple valves secured therein, and the venous stent may include various lengths sufficient to accommodate spacing of the multiple valves within the stent approximately 2 cm or more apart from one another.

In some embodiments, venous stents disclosed herein may be sized and dimensioned for treatment of other venous insufficiencies. For example, some embodiments of the venous stents disclosed herein may be sized and dimensioned to treat venous insufficiency in venous areas such as the greater or lesser saphenous veins, iliac veins, and common femoral vein, etc. In some embodiments, the venous stents disclosed herein are sized and dimensioned for treatment of May-Thurner Syndrome, such as venous stents having one or more valves secured therein and sized and dimension for implanting in the left iliac vein to reduce compression by the iliac artery and decrease pain from reduced blood flow. As iliac vein diameters can vary from about 10 mm to about 18 mm, some embodiments of the venous stents disclosed herein may include venous stents having one or more valves secured therein and a diameter of about 10 mm to about 20 mm.

Also disclosed herein are methods of manufacturing venous stents having one or more valves secured therein. In some embodiments, the venous stents may be manufactured by a multi-mandrel polytetrafluoroethylene (PTFE) or expanded PTFE (ePTFE)-lining process. In an example, the mandrels used in this method may be shaped to operate as a mold for the PTFE-lining process. In a more particular example, the mandrels used in this method may include one generally convex-shaped mandrel and one generally concave-shaped mandrel. A PTFE layer or material, which may ultimately form the valve (e.g., the valve PTFE) may be placed between the concave and convex surfaces of each mandrel. In an embodiment, the PTFE layer may be created by placing a PTFE layer between the mandrels or by sliding a sock-shaped PTFE sleeve over the mandrels. A liner of PTFE (e.g., the inner PTFE sleeve or layer) may be placed over the mandrel and the edges of the valve PTFE. A stent may then be loaded over the mandrels, and an outer sleeve of PTFE (e.g., the outer PTFE sleeve or layer) may be loaded over the stent. The assembly including the outer PTFE sleeve, the stent, the inner PTFE sleeve, and the valve PTFE may then be compressed on the mandrel and held during a sintering process to create the valve. The mandrels may then be unloaded from the stent openings, and a PTFE valve membrane would be left within a lumen formed from the removed mandrels, with the PTFE valve membrane adhered to the inner PTFE sleeve and the outer PTFE sleeve. The PTFE valve membrane in the lumen may then be cut through a portion of the PTFE valve membrane to create a passage through the valve.

The one or more valves in the venous stents disclosed herein are described in greater detail below and may include a native-style bi-cuspid valve, a single “pocket” valve, multiple valves, a “petal valve” that is layered in a petal like formation that opens and closes as blood flows through the valve, a “jelly fish” valve having a mushroom shape and including holes that the blood flows through, and/or an “oculus” or funnel valve that opens to allow blood to flow through. In many embodiments, the venous stent may include a tapered PTFE thickness, with the thickest portion of the PTFE near the wall of the device to allow better adhesion during a securement or bonding process. For example, in some embodiments, the thickness of the PTFE material may not be uniform throughout the entire valve. More specifically, the valve may have a thicker portion next to the stent wall to create a robust bond to the wall sleeve and then taper to a thinner flap that creates the interface between the seal and the valve. This tapering also can be tuned to give support to the valve to prevent prolapse (e.g., a medium thickness length in the middle of the valve). In some embodiments, the valves may include a thicker PTFE or a stiffer material to create varying stiffness of valves as needed.

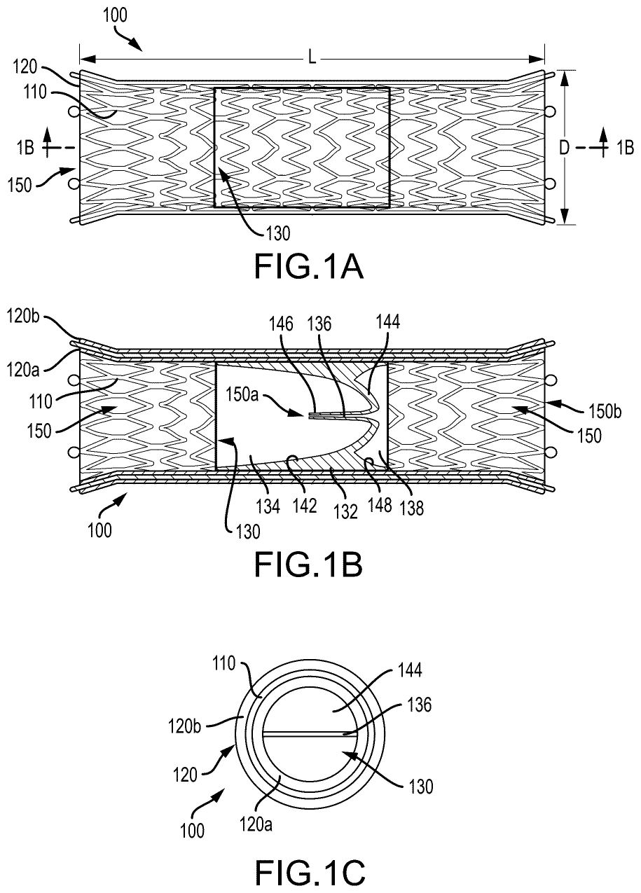

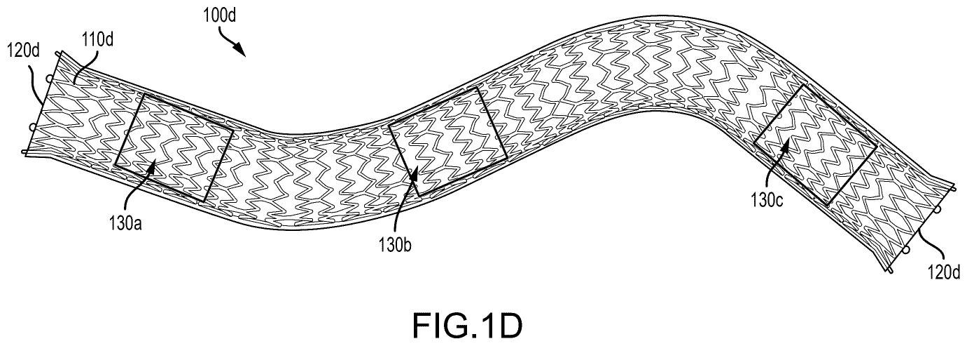

Turning to the drawings, FIG. 1A is a side view of a venous stent 100 having a valve 130 secured therein, FIG. 1B is a cross-sectional view of the venous stent 100 taken along plane 1B-1B, and FIG. 1C is a front view of the venous stent 100, according to an embodiment. The venous stent 100 may include a stent body 110 that is expandable and has a lumen 150 extending therethrough, a sleeve 120 secured to the stent body 110, and the valve 130 secured to the stent body and the sleeve 120 within the lumen 150. The stent body 110 may be self-expandable or expandable responsive to balloon deployment of the venous stent 100. In some embodiments, the stent body 110 may be formed from a nickel-titanium superelastic alloy (e.g., nitinol) or other superelastic material which allows the stent body 110 to self-expand and/or to deploy when an external compressive force is removed (e.g., the external container sheath is removed from the stent body 110, and the venous stent 100 can switch from the collapsed state to the deployed state). In some embodiments, such as a venous stent that is expandable responsive to a balloon deployment, the stent body 110 may include other material(s), such as stainless steel. Descriptions and aspects of stent bodies (e.g., tubular prosthesis) are disclosed in U.S. patent application Ser. No. 15/966,120, filed on Apr. 30, 2018, the disclosure of which is incorporated herein, in its entirety, by this reference.

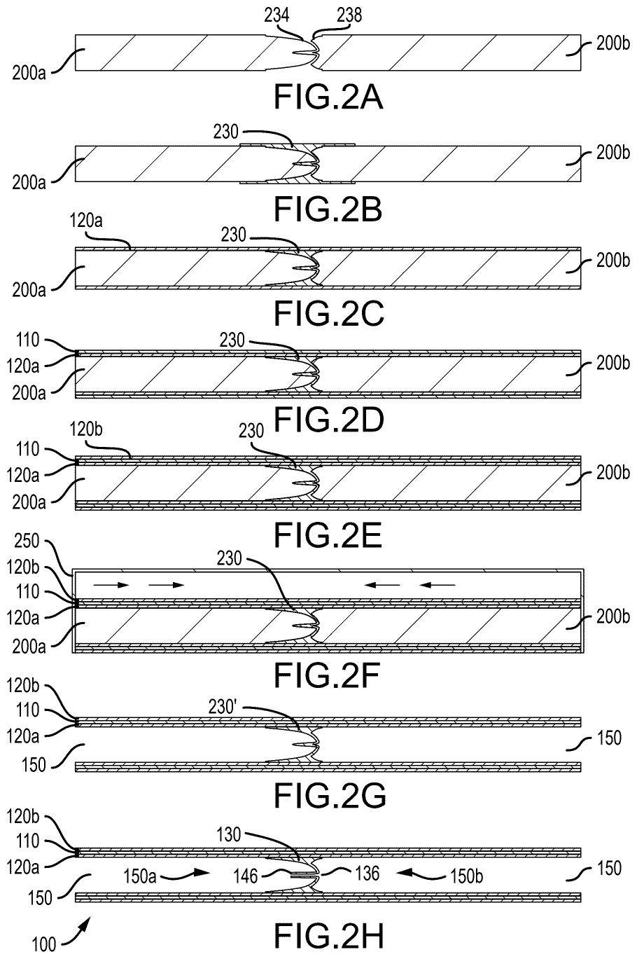

The sleeve 120 is secured to the stent body 110 and at least partially defines the lumen 150 extending through the stent body 110. In some embodiments, the stent body 110 is at least partially (e.g., entirely) embedded in the sleeve 120. For example, as described in greater detail below, the venous stent 100 may be formed by positioning the stent body 110 between an liner 120a and an outer sleeve 120b (shown in FIGS. 2D-2H) and then compressing and/or bonding the liner 120a and the outer sleeve 120b together with the stent body 110 positioned at least partially (e.g., entirely) therebetween. The liner 120a and the outer sleeve 120b may be bonded or adhered together during a variety of processes, such as ultrasonic bonding or welding, thermal bonding, sintering, etc. In some embodiments, the sleeve 120 forms a cover over the stent body 110. The material of the sleeve 120 may be configured to expand with the stent body 110 as the venous stent 100 is deployed. In some embodiments, the sleeve 120 may be formed from a PTFE material or a ePTFE material.

The valve 130 is secured to the sleeve 120 within the lumen 150, according to an embodiment. For example, the valve 130 may be bonded or adhered to the liner 120a of the sleeve 120. Accordingly, the valve 130 may be indirectly secured to the stent body 110 via the sleeve 120 (e.g., the liner 120a of the sleeve 120). In some embodiments, the valve 130 includes a material that is substantially identical to the material of the sleeve 120 (e.g., the liner 120a and/or the outer sleeve 120b of the sleeve 120). For example, the valve 130 and the sleeve 120 (e.g., the liner 120a and/or the outer sleeve 120b of the sleeve 120) may both include PTFE material. In other embodiments, the valve 130 may be formed from a material that is different than the material of the sleeve 120.

The valve 130 is configured to inhibit (e.g., partially restrict or substantially completely prevent) a first direction 150a of blood flow through the valve 130 in the lumen 150 and allow a second direction 150b of blood flow through the valve 130 in the lumen 150 when the venous stent 100 is implanted into the vein. In the venous stent 100, the valve 130 includes a bicuspid valve 130. However, as described herein, other valve configurations may be employed besides bicuspid valves. For example, the valve 130 may include a valve body 132 having a first region 142 (e.g., distal region) at least partially defining a first chamber 134 (e.g., distal chamber) and a second region 148 (e.g., proximal region) at least partially defining a second chamber 138 (e.g., proximal chamber). The first chamber 134 may be positioned to such that blood flowing in the first direction 150a enters the valve 130 in the first chamber 134. The second chamber 138 may be positioned such that blood flowing in the second direction 150b enters the valve 130 in the second chamber 138.

The valve 130 may include one or more regions positioned and shaped to allow blood flowing the in second direction 150b and/or in the second chamber 138 to pass through the valve 130, while inhibiting or substantially prevent blood flowing in the first direction 150a and/or in the first chamber 134 from passing through the valve 130. For example, the valve 130 may include opposing inwardly extending regions 144 positioned between and at least partially defining the first chamber 134 and the second chamber 138. The inwardly extending regions 144 of the valve 130 may arch or angle inward and proximally from the first region 134 towards the second region 148 of the valve 130. The valve 130 also may include opposing walls 146 defining an opening 136 that extends through the valve 130 that allows blood to flow therethrough in second direction 150b. The opposing walls 146 may extend distally from the inwardly extending region 144 into the first chamber 134. In some embodiments, the opening 136 is an elongated opening 136 that narrows as the elongated opening progresses distally (e.g., moves further into the first chamber 134). Accordingly, the opposing walls 146 may move closer together as the opposing walls 144 extend distally further into the first chamber 134. In some embodiments, the opening 136 extends across substantially all of the lumen 150. Once implanted, blood flowing in the first direction 150a and into the first chamber 134 may exert a force on the inwardly extending regions 144 that pushes the opposing walls 146 together and substantially closes the opening 146. In contrast, blood flowing in the second direction 150b may pass through the opening 136.

The venous stent 100 may be sized and dimensioned to be implanted in veins of varying sizes to inhibit blood flow through the valve 130 in the first direction 150a in the vein and allow blood flow through the valve 130 in the second direction 150b in the vein. For example, the venous stent 100 may be sized and dimensioned to be implanted into an ovarian vein and may include a diameter D of about 1 mm to about 8 mm, such as a diameter D of about 3 mm to about 4 mm for a normal or typical ovarian vein, a diameter D of about 5 mm to about 6 mm for a nominally dilated ovarian vein, or a diameter D of about 7 mm to about 8 mm for a grossly dilated ovarian vein. In some embodiments, the venous stent 100 is sized and dimensioned to be implanted into an iliac vein and has a diameter of about 10 mm to about 18 mm.

The length L of the venous stent 100 may vary according to different embodiments and different purposes. For example, the venous stent 100 illustrated in FIGS. 1A-1C may include a single valve 130, and the venous stent 100 may have a length L of about 10 mm or more. For example, the venous stent 100 may include a length L of about 10 mm to about 100 mm, about 10 mm to about 50 mm, about 10 mm to about 40 mm, about 10 mm to about 30 mm, about 10 mm to about 20 mm, or about 10 mm to about 15 mm.

In some embodiments, a venous stent 100 according to this disclosure includes a plurality of valves 130 within a single venous stent 100. For example, turning ahead in the drawings, FIG. 1D is a side view of a venous stent 100d having a plurality of valves 130a-c secured therein, according to an embodiment. Unless otherwise noted, the venous stent 100d, the stent body 110d, the sleeve 120d, and the valves 130a-c may include any aspect of the venous stent 100, the stent body 110, the sleeve 120, and the valve 130. While the venous stent 100d includes three valves 130a-c, other embodiments of the venous stent 100d may include other numbers of valves 130 (e.g., two, three, four, or more). The plurality of valves 130a-c may be spaced within the lumen 150 of the venous stent 100 d at least about 2 cm from the adjacent valve of the plurality of valves 130a, 130b, 130c. Embodiments of the venous stent 100d having a plurality of valves 130 may have a length of about 4 cm to about 20 cm, about 4 cm to about 12 cm, about 12 cm to about 20 cm, about 4 cm to about 8 cm, about 8 cm to about 12 cm, about 12 cm to about 16 cm, or about 16 cm to about 20 cm.

Turning ahead in the drawings, FIGS. 2A-2H are side cross-sectional views of the venous stent 100 of FIG. 1A during a method or process of manufacture of the venous stent 100, according to an embodiment. The venous stent 100 may be manufactured by a multi-mandrel lining (e.g., PTFE lining) process. As illustrated in FIG. 2A, the ends 234, 238 of two mandrels 200a, 200b are positioned proximate to each other. The mandrels 200a, 200b may be shaped to be a mold for the lining process. Accordingly, the ends of the mandrels 200a, 200b may be shaped to form the valve 130 between and/or around the ends of the mandrels 200a, 200b. For example, the first mandrel 200a may have an end 234 that is generally convex to generally form the first chamber 134 of the valve 130, and the second mandrel 200b may have an end 238 that is generally concave to form the second chamber 138 of the valve 130. The end 234 of the first mandrel 200a also may include a slot 246 positioned to form the walls 146 of the valve 130, and the end 238 of the second mandrel 200b may include protrusion or point 236 positioned to extend at least partially into the slot 246 when the end 238 of the second mandrel 200b is disposed proximate to the end 238 of the first mandrel 200a.

As illustrated in FIG. 2B, valve material 230 (e.g., PTFE) is disposed between the ends 234, 238 of the two mandrels 200a, 200b. The valve material 230 may be referred to as a PTFE layer or valve PTFE. The valve material 230 may be placed between the mandrels 200a, 200b or by sliding an initial sleeve (e.g., PTFE sleeve) over the end regions of the two mandrels 200a, 200b to cover the ends 234, 238 positioned proximate to one another.

As illustrated in FIG. 2C, a first or liner 120a (e.g., inner PTFE sleeve) may be placed over the valve material 230 (e.g., over the edges of the valve material 230) and the mandrels 200a, 200b. As illustrated in FIG. 2D, the stent 110 may be loaded or positioned over the liner 120a (and the valve material 230 and the mandrels 200a, 200b). As illustrated in FIG. 2E a second or outer sleeve 120b (e.g., outer PTFE sleeve) may be placed over the stent 110 (and the liner 120a, the valve material 230, and the mandrels 200a, 200b).

Turning to FIG. 2F, the assembly of the outer sleeve 120b, the stent 110, the liner 120a, and the valve material 230 disposed on the mandrels 200a, 200b may be placed into a compression fixture 250 and held during a securement process to create a valve membrane 230'. This securement process may include a bonding or adhesion process configured to secure (e.g., bond) the valve material 230 to the liner 120a and secure (e.g., bond) the liner 120a to the outer sleeve 120b with the stent body 110 at least partially therebetween. Accordingly, the stent body 110 may be at least partially (e.g., entirely) embedded within the sleeve 120, and the valve membrane 230′ (and subsequent valve 130) may be indirectly secured to the stent body 110 through the liner 120a of the sleeve. The liner 120a and the outer sleeve 120b may be bonded or adhered together during a variety of processes, such as ultrasonic bonding or welding, thermal bonding, sintering, etc.

As illustrated in FIG. 2G, the mandrels 200a, 200b may be removed to form the lumen 150 and leave the valve membrane 230′ exposed. In some embodiments, the opening 136 of the valve 130 is not present or formed in the valve membrane 230′ during the molding and sintering process. Accordingly, in some embodiments and as illustrated in FIG. 2H, the valve membrane 230′ may be cut between the walls 146 (e.g., cut approximately central through valve membrane 230') to form the opening 136 between the walls 146 and finalize the valve 130. This process of manufacturing the venous stent 100 results in a floating valve 130 secured to the stent body 110 of the venous stent 100.

Unless otherwise noted, embodiments of a method or process of manufacture of the venous stent 100d having multiple valves 130a-c may include any aspect shown in FIGS. 2A-2H in the method or process of manufacture of the venous stent 100. In manufacturing the venous stent 100d, four mandrels may be used to form the three valves 130a-c. The outermost mandrel(s) may be removed first, and the center mandrel(s) may be removed through the valve membrane 230′ or the finished valve 130a-c after the valve has been cut to form the opening 136.

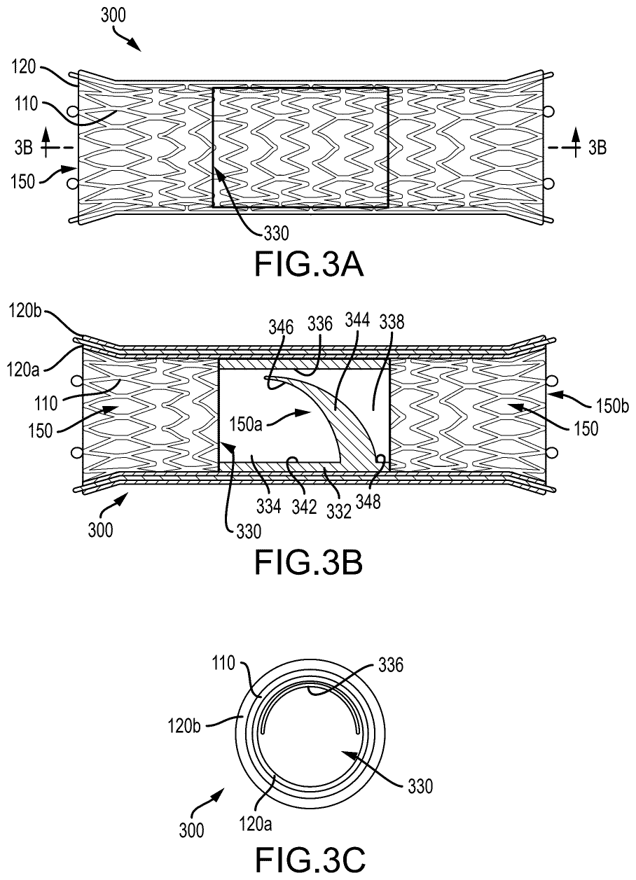

Turning ahead in the drawings, FIG. 3A is a side view of a venous stent 300 having a valve 330 secured therein, FIG. 3B is a cross-sectional view of the venous stent 300 taken along plane 3B-3B, and FIG. 3C is a front view of the venous stent 300, according to an embodiment. Unless otherwise noted, the venous stent 300 may include any aspect of the venous stent 100. For example, the venous stent 300 includes a stent body 110, a sleeve 120, and the valve 330 that may include any aspect (e.g., materials, shapes, configurations, dimensions) of the stent body 110, the sleeve 120, and the valve 130 unless otherwise noted herein. Moreover, although the venous stent 300 is shown with a single valve 330, other embodiments of the venous stent 300 may include a plurality of valves 330, similar to the venous stent 100d. Furthermore, some embodiments of venous stents disclosed herein may include a plurality of valves, one or more of which is the valve 130 and one or more of which is the valve 330.

The valve 330 is secured to the stent body 110 and the sleeve 120 within the lumen 150, according to an embodiment. For example, the valve 330 may be bonded or adhered to the liner 120a of the sleeve 120. Accordingly, the valve 330 may be indirectly secured to the stent body 110 via the sleeve 120 (e.g., the liner 120a of the sleeve 120). In some embodiments, the valve 330 includes a material that is substantially identical to the material of the sleeve 120 (e.g., the liner 120a and/or the outer sleeve 120b of the sleeve 120). For example, the valve 330 and the sleeve 120 (e.g., the liner 120a and/or the outer sleeve 120b of the sleeve 120) may both include PTFE material. In other embodiments, the valve 330 may be formed from a material that is different than the material of the liner 120a and/or the sleeve 120b.

The valve 330 is configured to inhibit a first direction 150a of blood flow through the valve 130 in the lumen 150 and allow a second direction 150b of blood flow through the valve 330 in the lumen 150 when the venous stent 300 is implanted into the vein. In the venous stent 300, the valve 330 includes a pocket valve 330. For example, the valve 330 may include a valve body 332 having a first region 342 (e.g., distal region) at least partially defining a first chamber 334 (e.g., distal chamber) and a second region 348 (e.g., proximal region) at least partially defining a second chamber 338 (e.g., proximal chamber). The first chamber 334 may be positioned to such that blood flowing in the first direction 150a enters the valve 330 in the first chamber 334. The second chamber 338 may be positioned such that blood flowing in the second direction 150b enters the valve 330 in the second chamber 338.

The valve 330 may include one or more regions positioned and shaped to allow blood flowing in the second direction 150b and/or in the second chamber 338 to pass through the valve 130, while inhibiting or substantially prevent blood flowing in the first direction 150a and/or in the first chamber 334 from passing through the valve 330. For example, the valve 130 may include a wall 344 that extends distally from a portion of the valve body 332 before terminating at a terminating region 346 that defines an opening 336 extending through the valve 330. The wall 344 may define a substantially concave or pocket portion of the first chamber 334. The opening 336 may be arched such that the opening 336 forms a semi-or half-circle, as shown in FIG. 3C. In some embodiments, the opening 336 also is an elongated opening 336 that narrows as the elongated opening progresses distally (e.g., moves further into the first chamber 334). Once implanted, blood flowing in the first direction 150a and into the first chamber 334 may exert a force on the wall 344 that pushes the wall 334 against the valve body 332 and substantially closes the opening 346. In contrast, blood flowing in the second direction 150b may pass through the opening 336.

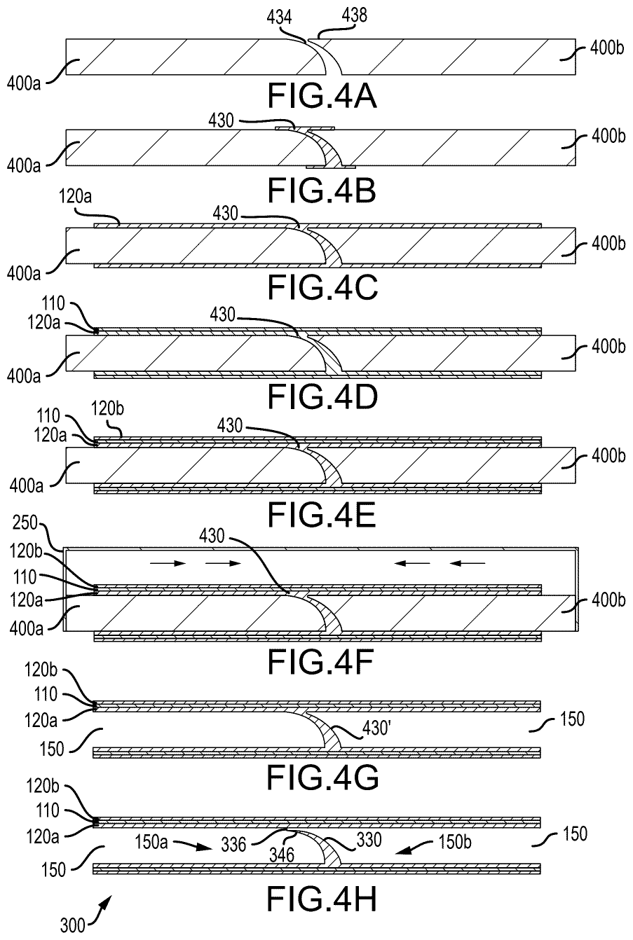

Turning ahead in the drawings, FIGS. 4A-4H are side cross-sectional views of the venous stent 300 of FIG. 3A during a method or process of manufacture of the venous stent 300, according to an embodiment. The venous stent 300 may be manufactured by a multi-mandrel lining (e.g., PTFE lining) process. As illustrated in FIG. 3A, the ends 434, 438 of two mandrels 400a, 400b are positioned proximate to each other. The mandrels 400a, 400b may be shaped to be a mold for the lining process. Accordingly, the ends of the mandrels 400a, 400b may be shaped to form the valve 330 between and/or around the ends of the mandrels 400a, 400b. For example, the first mandrel 400a may have an end 434 that is generally partially convex or semispherical to generally form the first chamber 434 of the valve 430, and the second mandrel 400b may have an end 438 that is generally partially concave to form the second chamber 338 of the valve 330.

As illustrated in FIG. 4B, valve material 430 (e.g., PTFE) is disposed between the ends 434, 438 of the two mandrels 400a, 400b. The valve material 430 may be referred to as a PTFE layer or valve PTFE. The valve material 430 may be placed between the mandrels 400a, 400b or by sliding an initial sleeve (e.g., PTFE sleeve) over the end regions of the two mandrels 400a, 400b to cover the ends 434, 438 positioned proximate to one another.

As illustrated in FIG. 4C, a first or liner 120a (e.g., inner PTFE sleeve) may be placed over the valve material 430 (e.g., over the edges of the valve material 230) and the mandrels 400a, 400b. As illustrated in FIG. 4D, the stent 110 may be loaded or positioned over the liner 120a (and the valve material 430 and the mandrels 400a, 400b). As illustrated in FIG. 4E a second or outer sleeve 120b (e.g., outer PTFE sleeve) may be placed over the stent 110 (and the liner 120a, the valve material 430, and the mandrels 400a, 400b).

Turning to FIG. 4F, the assembly of the outer sleeve 120b, the stent 110, the liner 120a, and the valve material 430 disposed on the mandrels 400a, 400b may be placed into a compression fixture 250 and held during a securement process to create a valve membrane 430'. This securement process may include a bonding or adhesion process configured to secure (e.g., bond) the valve material 430 to the liner 120a and secure (e.g., bond) the liner 120a to the outer sleeve 120b with the stent body 110 at least partially therebetween. Accordingly, the stent body 110 may be at least partially (e.g., entirely) embedded within the sleeve 120, and the valve membrane 430′ (and subsequent valve 330) may be indirectly secured to the stent body 110 through the liner 120a of the sleeve. The liner 120a and the outer sleeve 120b may be bonded or adhered together during a variety of processes, such as ultrasonic bonding or welding, thermal bonding, sintering, etc.

As illustrated in FIG. 4G, the mandrels 400a, 400b may be removed to form the lumen 150 and leave the valve membrane 430′ exposed. In some embodiments, the opening 336 of the valve 330 is not present or formed in the valve membrane 430′ during the molding and sintering process. Accordingly, in some embodiments and as illustrated in FIG. 4H, the valve membrane 430′ may be cut between the valve body 332 and the terminating region 346 of the wall 344 (e.g., cut in a semi-or half-circle through valve membrane 430') to form the opening 336 between the terminating region 346 of the wall 344 and finalize the valve 330. This process of manufacturing the venous stent 300 results in a floating valve 330 secured to the stent body 110 of the venous stent 300.

Unless otherwise noted, embodiments of a method or process of manufacture of a venous stent having multiple valves 330 may include any aspect shown in FIGS. 4A-4H in the method or process of manufacture of the venous stent 300. In manufacturing the venous stent having multiple valves 330, three or more mandrels may be used to form the multiple valves 330. The outermost mandrel(s) may be removed first, and the center mandrel(s) may be removed through the valve membrane 430′ or the finished valve 330 after the valve has been cut to form the opening 336.

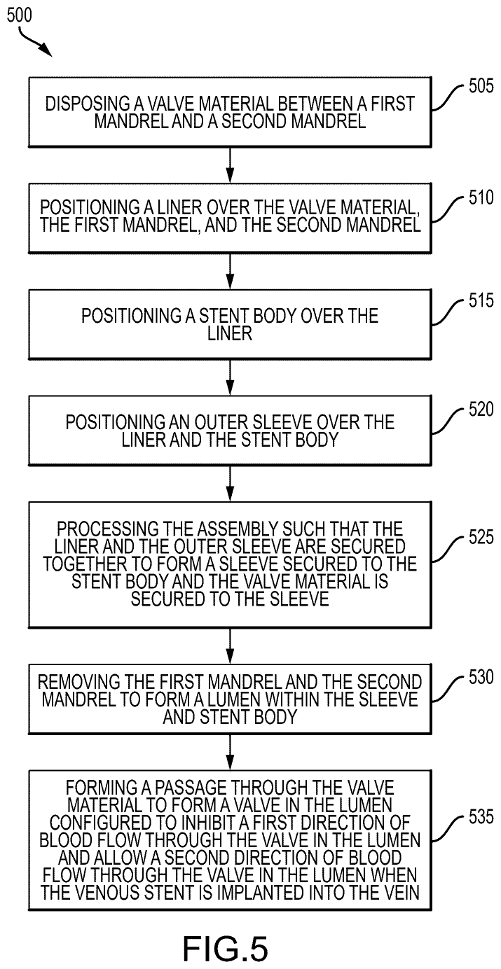

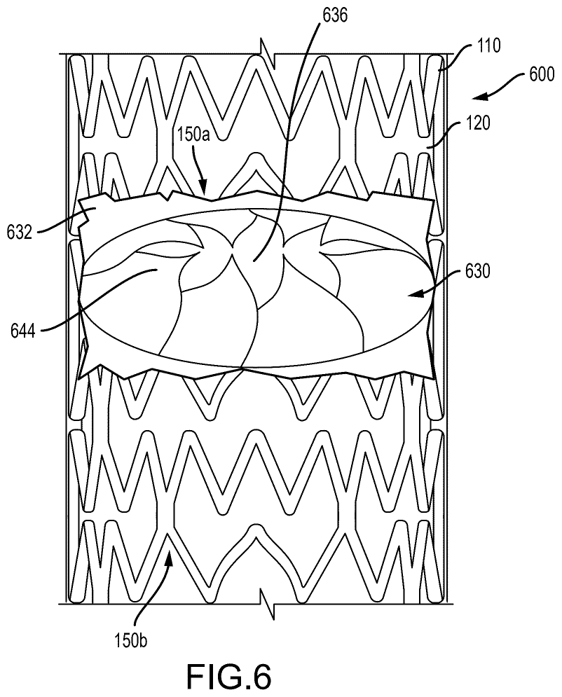

Turning ahead in the drawings, FIG. 5 is a flow diagram of a method 500 of manufacturing a venous stent having a valve secured therein, according to an embodiment. The venous stents manufactured according to the method 500 may include any venous stent disclosed herein, such as but not limited to the venous stents 100, 100d, 300 described above, and the venous stents 600, 700, 800 described below. In many embodiments, the method 500 encompasses the methods and processes of manufacture of the venous stents described above and shown in FIGS. 2A-2H and FIGS. 4A-4H. Accordingly, the method 500 of forming a venous stent may include disposing 505 a valve material between a first mandrel and a second mandrel, and positioning 510 a liner over the valve material, the first mandrel, and the second mandrel. The method 500 also may include positioning 515 a stent body over the liner and positioning 520 an outer sleeve over the liner to form an assembly. The method 500 also may include compressing and sintering 525 the assembly such that the liner and the outer sleeve are secured together to form a sleeve secured to the stent body and the valve material is secured to the sleeve and the stent body. The method 500 also may include removing 530 the first mandrel and the second mandrel to form a lumen within the sleeve and the stent body. The method also may include forming 535 a passage or opening through the valve material to form a valve in the lumen configured to inhibit a first direction of blood flow through the valve in the lumen and allow a second direction of blood flow through the valve in the lumen when the venous stent is implanted into the vein.

In some embodiments, the venous stent manufactured according to the method 500 is sized and dimensioned to be implanted into an ovarian vein and has a diameter of about 1 mm to about 8 mm. In some embodiments, the venous stent manufactured according to the method 500 is sized and dimensioned to be implanted into an iliac vein and has a diameter of about 10 mm to about 18 mm. In some embodiments of the method 500, the valve material, the liner, and the outer sleeve include a substantially identical material.

In some embodiments, the method 500 also includes disposing additional valve material between the second mandrel and a third mandrel such that the additional valve material is spaced at least about 2 cm from the valve material. In these and other embodiments, positioning 510 a liner over the valve material, the first mandrel, and the second mandrel may include positioning the liner over the valve material, the additional valve material, the first mandrel, and the second mandrel. In these and other embodiments, compressing and sintering 525 the assembly may include compressing and sintering the assembly such that the valve material and the additional valve material are secured to the sleeve and the stent body. In these and other embodiments, removing 530 the first mandrel and the second mandrel to form a lumen within the sleeve and the stent body includes removing the first mandrel, the second mandrel, and the third mandrel to form the lumen within the sleeve and the stent body. In some embodiments, one or more of the mandrels may be removed through one or more of the valves. The method 500 also may include forming an additional passage through the additional valve material to form an additional valve in the lumen configured to inhibit the first direction of blood flow through the valve in the lumen and allow the second direction of blood flow through the additional valve in the lumen when the venous stent is implanted into the vein.

In some embodiments of the method 500, disposing 505 a valve material between a first mandrel and a second mandrel may include disposing the valve material between an at least partially concave end of the first mandrel and an at least partially convex end of the second mandrel, with the valve being a bicuspid valve. In some embodiments of the method 500, disposing 505 a valve material between a first mandrel and a second mandrel may include disposing the valve material between an at least partially concave end of the first mandrel and an at least partially convex end of the second mandrel, with the valve being a pocket valve.

Other embodiments of venous stents having a valve secured within the lumen are disclosed herein. Turning ahead in the drawings, FIG. 6 is a side view of a venous stent 600 having a stent body 110, a sleeve 120, and a valve 630 secured therein, according to an embodiment. In FIG. 6, a portion of the sleeve 120 and the stent body 110 is removed for viewing of the valve 630. Unless otherwise noted, the venous stent 600 may include any aspect of the venous stent 100, 100d, 300. For example, the venous stent 600 includes the stent body 110, the sleeve 120, and the valve 630 that may include any aspect (e.g., materials, shapes, configurations, dimensions) of the stent body 110, the sleeve 120, and the valve 130, 330, unless otherwise noted herein. Moreover, although the venous stent 600 is shown with a single valve 630, other embodiments of the venous stent 600 may include a plurality of valves 630, similar to the venous stent 100d. Furthermore, some embodiments of venous stents disclosed herein may include a plurality of valves having any combination of the valve 130, the valve 330, and/or the valve 630.

The valve 630 is secured to the stent body 110 and the sleeve 120 within the lumen 150, according to an embodiment. In some embodiments, the valve 630 includes a material that is substantially identical to the material of the sleeve 120. For example, the valve 630 and the sleeve 120 may both include PTFE material. In other embodiments, the valve 630 may be formed from a material that is different than the material of the sleeve 120.

The valve 630 is configured to inhibit a first direction 150a of blood flow through the valve 630 in the lumen 150 and allow a second direction 150b of blood flow through the valve 630 in the lumen 150 when the venous stent 600 is implanted into the vein. In the venous stent 600, the valve 630 includes a petal valve 630. The petal valve 630 may include a plurality of movable petals 644 secured to a valve body 632 and configured to inhibit the first direction 150a of blood flow through the lumen and allow the second direction 150b of blood flow through the lumen 150 when the venous stent 600 is implanted into the vein. For example, the valve 630 may be layered with the plurality of petals 644 in a petal-like configuration where each petal overlaps one adjacent petal is overlapped by another adjacent petal. This configuration allows the plurality of petals 644 to close the opening 636 when blood flows in the first direction 150a, and allows the plurality of petals 644 to open the opening 636 when the blood flows in the second direction 150b. The plurality of petals 644 may be formed from expanded PTFE (ePTFE) or a polycarbonate-urethane.

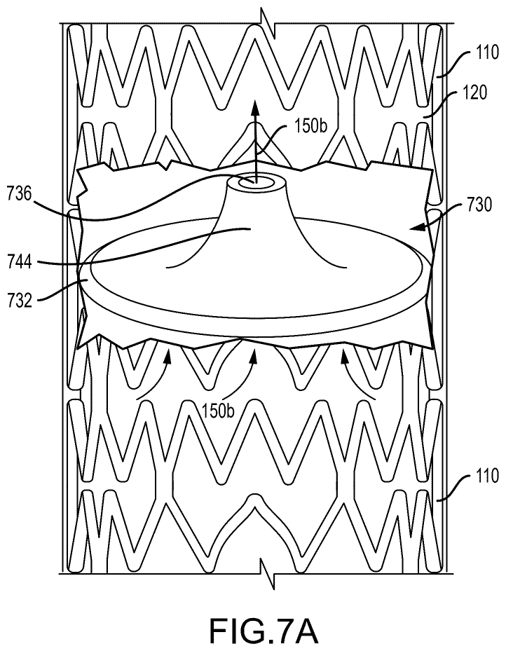

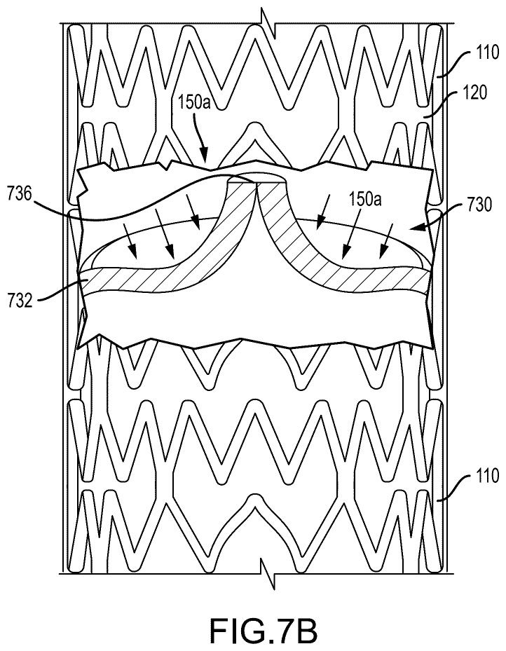

FIG. 7A is a cutaway side view of a venous stent 700 having a valve 730 in an open configuration secured therein, and FIG. 7B is a cutaway side partial cross-sectional view of the venous stent 700 having the valve 730 in closed configuration, according to an embodiment. The venous stent 700 also may include a stent body 110 and a sleeve 120. In FIGS. 7A-7B, a portion of the sleeve 120 and the stent body 110 is removed for viewing of the valve 730. Unless otherwise noted, the venous stent 700 may include any aspect of the venous stent 100, 100d, 300, 600. For example, the venous stent 700 includes the stent body 110, the sleeve 120, and the valve 730 that may include any aspect (e.g., materials, shapes, configurations, dimensions) of the stent body 110, the sleeve 120, and the valve 130, 330, 630 unless otherwise noted herein. Moreover, although the venous stent 700 is shown with a single valve 730, other embodiments of the venous stent 700 may include a plurality of valves 730, similar to the venous stent 100d. Furthermore, some embodiments of venous stents disclosed herein may include a plurality of valves having any combination of the valve 130, the valve 330, the valve 630, and/or the valve 730.

The valve 730 is secured to the stent body 110 and the sleeve 120 within the lumen 150, according to an embodiment. In some embodiments, the valve 730 includes a material that is substantially identical to the material of the sleeve 120. For example, the valve 730 and the sleeve 120 may both include PTFE material. In other embodiments, the valve 730 may be formed from a material that is different than the material of the sleeve 120.

The valve 730 is configured to inhibit a first direction 150a of blood flow through the valve 730 in the lumen 150 (shown in FIG. 7B) and allow a second direction 150b of blood flow through the valve 730 in the lumen 150 (shown in FIG. 7A) when the venous stent 700 is implanted into the vein. In the venous stent 700, the valve 730 includes a funnel valve 730. The funnel valve 730 may include a funnel portion 744 secured to a valve body 732 and configured to inhibit the first direction 150a of blood flow through the lumen (shown in FIG. 7B) and allow the second direction 150b of blood flow through the lumen 150 (shown in FIG. 7A) when the venous stent 700 is implanted into the vein. For example, the funnel portion 744 of the valve 730 may define an opening 736 that closes when blood flows in the first direction 150a and opens when the blood flows in the second direction 150b. More particularly, as shown in FIG. 7A, blood flowing in the second direction 150b may push up on the opening 736, thereby allowing the flow of blood in the second direction 150b through the opening 736 in the valve 730. With specific reference to FIG. 7B, when the lower part of the vein depressurizes, the weight of the blood in the first direction 150a in the upper part may close the opening 736, thereby inhibiting the flow of blood in the first direction 150a through the valve 730 (e.g., inhibiting reflux).

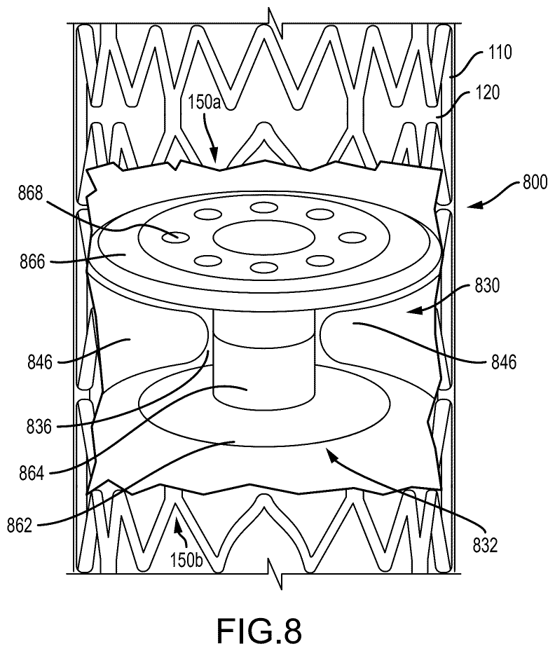

FIG. 8 is a side view of a venous stent 800 having a stent body 110, a sleeve 120, and a valve 830 secured therein, according to an embodiment. In FIG. 8, a portion of the sleeve 120 and the stent body 110 is removed for viewing of the valve 830. Unless otherwise noted, the venous stent 800 may include any aspect of the venous stent 100, 100d, 300, 600, 700. For example, the venous stent 800 includes the stent body 110, the sleeve 120, and the valve 830 that may include any aspect (e.g., materials, shapes, configurations, dimensions) of the stent body 110, the sleeve 120, and the valve 130, 330, 630, 730, unless otherwise noted herein. Moreover, although the venous stent 800 is shown with a single valve 830, other embodiments of the venous stent 800 may include a plurality of valves 830, similar to the venous stent 100d. Furthermore, some embodiments of venous stents disclosed herein may include a plurality of valves having any combination of the valve 130, the valve 330, the valve 630, the valve 730, and/or the valve 830.

The valve 830 is at least partially secured to the stent body 110 and the sleeve 120 within the lumen 150, according to an embodiment. In some embodiments, the valve 830 includes a material that is substantially identical to the material of the sleeve 120. For example, the valve 830 and the sleeve 120 may both include PTFE material. In other embodiments, the valve 830 may be formed from a material that is different than the material of the sleeve 120.

The valve 830 is configured to inhibit a first direction 150a of blood flow through the valve 830 in the lumen 150 and allow a second direction 150b of blood flow through the valve 830 in the lumen 150 when the venous stent 800 is implanted into the vein. In the venous stent 800, the valve 830 includes a valve seat 846 defining a channel 836 extending therethrough and a valve body 832. The valve body 832 may be generally mushroom-shaped and may be movable within the valve 830. In some embodiments, the valve body 832 includes a head 866 having a plurality of holes 868, a neck 864, and a base 862. The neck 864 is positioned on the valve body 832 between the base 862 and the head 866. The neck 864 also may be positioned within the channel 836 defined by the one or more valve seats 846. In some embodiments, the neck 864 includes a length greater than a length of the one or more valve seats 846, while the head 866 has a greater diameter than the channel 836 defined by the one or more seats 846. The head 866 also may have a greater diameter than the base 862.

When the venous stent 800 is implanted into the vein, blood flowing in the second direction 150b flows through the passthrough holes 868, lifting the valve body 832 and allowing the blood flow in the second direction 150b through the valve 830. When pressure below the valve 830 is reduced, the valve body 832 may reseat on the valve seat 844, thereby inhibiting the flow of blood in the first direction 150a. In some embodiments, the venous stent 830 may include multiple valve seats 846, and the head 866 of the valve body 832 may be positioned between adjacent valve seats 846. The valve 830 is configured to lift with a small amount of pressure in the blood stream to allow the flow of blood in the second direction 150b but without the base 862 inhibiting the flow of blood in the second direction 150b. The base 150b also may be configured to prevent excessive or undesired travel of the valve 830 in the blood stream.

Also disclosed herein is a method of inhibiting venous reflux flow and chronic venous insufficiency. The method comprises implanting into a vein any of the venous stents disclosed herein. For example, the venous stent implanted into the vein according to the method may include a stent body that is expandable and has a lumen extending therethrough, a sleeve secured to the stent body and at least partially defining the lumen extending through the stent body, and a valve secured to the stent body and the sleeve within the lumen. The valve allows a first direction of blood flow through the valve in the lumen and inhibits a second direction of blood flow through the valve in the lumen after the venous stent is implanted into the vein according to the method.

In some embodiments of the method of inhibiting venous reflux flow and chronic venous insufficiency, the vein having the venous stent implanted therein is an ovarian vein and the venous stent has a diameter of about 1 mm to about 8 mm. In some embodiments of the method of inhibiting venous reflux flow and chronic venous insufficiency, the vein having the venous stent implanted therein is an iliac vein and has a diameter of about 10 mm to about 18 mm.

In some embodiments of the method of inhibiting venous reflux flow and chronic venous insufficiency, the venous stent includes an additional valve secured to the stent body and the sleeve within the lumen. The additional valve may be spaced within the lumen at least about 2 cm from the valve, with the additional valve also allowing the first direction of blood flow through the valve in the lumen and inhibiting the second direction of blood flow through the valve in the lumen.

In some embodiments of the method of inhibiting venous reflux flow and chronic venous insufficiency, the valve and the sleeve include a substantially identical material. The valve(s) in the venous stent of the method may include one or more of a bicuspid valve, a pocket valve, a petal valve, a funnel valve, and/or any other valves disclosed herein.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A venous stent, comprising:
a stent body that is expandable and has a lumen extending therethrough, the stent body being sized and dimensioned to be implanted into a vein;
a sleeve secured to the stent body and at least partially defining the lumen extending through the stent body; and
a valve secured to the sleeve within the lumen, the valve configured to inhibit a first direction of blood flow through the valve in the lumen and allow a second direction of blood flow through the valve in the lumen when the venous stent is implanted into the vein.

2. The venous stent of claim 1, wherein the venous stent is sized and dimensioned to be implanted into an ovarian vein and has a diameter of about 1 mm to about 8 mm.

3. The venous stent of claim 1, wherein the venous stent is sized and dimensioned to be implanted into an iliac vein and has a diameter of about 10 mm to about 18 mm.

4. The venous stent of claim 1, further comprising an additional valve secured to the stent body and the sleeve within the lumen, the additional valve being spaced within the lumen at least about 2 cm from the valve and being configured to inhibit the first direction of blood flow through the valve in the lumen and allow the second direction of blood flow through the valve in the lumen when the venous stent is implanted into the vein.

5. The venous stent of claim 1, wherein the valve and the sleeve include a substantially identical material.

6. The venous stent of claim 1, wherein the valve includes a bicuspid valve.

7. The venous stent of claim 1, wherein the valve includes a pocket valve.

8. The venous stent of claim 1, wherein the valve includes a petal valve having a plurality of movable petals configured to inhibit the first direction of blood flow through the lumen and allow the second direction of blood flow through the lumen when the venous stent is implanted into the vein.

9. The venous stent of claim 1, wherein the valve includes a funnel valve.

10. The venous stent of claim 1, wherein the valve includes one or more valve seats defining a channel therethrough and a valve body having a head having a plurality of holes, a neck, and a base, the neck being positioned within the channel defined by the one or more valve seats and the head having a greater diameter than the base.

11. The venous stent of claim 1, wherein the sleeve includes a liner bonded to an outer sleeve, the valve being bonded to the liner.

12. The venous stent of claim 11, wherein the stent body is embedded within the sleeve between the liner and the outer sleeve.

13. A method of forming a venous stent, the method comprising:
disposing a valve material between a first mandrel and a second mandrel;
positioning a liner over the valve material, the first mandrel, and the second mandrel;
positioning a stent body over the liner;
positioning an outer sleeve over the liner to form an assembly;
processing the assembly such that the liner and the outer sleeve are secured together to form a sleeve secured to the stent body and the valve material is secured to the sleeve;
removing the first mandrel and the second mandrel to form a lumen within the sleeve and the stent body; and
forming a passage through the valve material to form a valve in the lumen configured to inhibit a first direction of blood flow through the valve in the lumen and allow a second direction of blood flow through the valve in the lumen when the venous stent is implanted into the vein.

14. The method of claim 13, wherein the venous stent is sized and dimensioned to be implanted into an ovarian vein and has a diameter of about 1 mm to about 8 mm.

15. The method of claim 13, wherein the venous stent is sized and dimensioned to be implanted into an iliac vein and has a diameter of about 10 mm to about 18 mm.

16. The method of claim 13, further comprising:
disposing additional valve material between the second mandrel and a third mandrel such that the additional valve material is spaced at least about 2 cm from the valve material;
wherein:
positioning a liner over the valve material, the first mandrel, and the second mandrel includes positioning the liner over the valve material, the additional valve material, the first mandrel, and the second mandrel;
processing the assembly includes processing the assembly such that the valve material and the additional valve material are secured to the sleeve; and
removing the first mandrel and the second mandrel to form a lumen within the sleeve and the stent body includes removing the first mandrel, the second mandrel, and the third mandrel to form the lumen within the sleeve and the stent body; and
forming an additional passage through the additional valve material to form an additional valve in the lumen configured to inhibit the first direction of blood flow through the valve in the lumen and allow the second direction of blood flow through the additional valve in the lumen when the venous stent is implanted into the vein.

17. The method of claim 13, wherein the valve material, the liner, and the outer sleeve include a substantially identical material.

18. The method of claim 13, wherein disposing a valve material between a first mandrel and a second mandrel includes disposing the valve material between an at least partially concave end of the first mandrel and an at least partially convex end of the second mandrel, and wherein the valve includes a bicuspid valve.

19. The method of claim 13, wherein disposing a valve material between a first mandrel and a second mandrel includes disposing the valve material between an at least partially concave end of the first mandrel and an at least partially convex end of the second mandrel, and wherein the valve includes a pocket valve.

20. The method of claim 13, wherein processing the assembly such that the liner and the outer sleeve are secured together to form a sleeve secured to the stent body and the valve material is secured to the sleeve includes bonding the outer sleeve to the liner to form the sleeve and bonding the valve material to the liner of the sleeve.

21. The method of claim 20, wherein bonding to the outer sleeve to the liner to form the sleeve includes bonding the outer sleeve to the liner to form the sleeve with the stent body positioned therebetween such that the stent body is at least partially embedded in the sleeve.

22. A method of inhibiting venous reflux flow and chronic venous insufficiency, the method comprising:
implanting a venous stent into a vein, the venous stent comprising:
a stent body that is expandable and has a lumen extending therethrough;
a sleeve secured to the stent body and at least partially defining the lumen extending through the stent body; and
a valve secured to the sleeve within the lumen, the valve allowing a first direction of blood flow through the valve in the lumen and inhibiting a second direction of blood flow through the valve in the lumen.

23. The method of claim 22, wherein the vein is an ovarian vein and the venous stent has a diameter of about 1 mm to about 8 mm.

24. The method of claim 22, wherein the vein is an iliac vein and has a diameter of about 10 mm to about 18 mm.

25. The method of claim 22, wherein the venous stent further includes an additional valve secured to the stent body and the sleeve within the lumen, the additional valve being spaced within the lumen at least about 2 cm from the valve and allowing the first direction of blood flow through the valve in the lumen and inhibiting the second direction of blood flow through the valve in the lumen.

26. The method of claim 22, wherein the valve and the sleeve include a substantially identical material.

27. The method of claim 22, wherein the valve includes a bicuspid valve.

28. The method of claim 22, wherein the valve includes a pocket valve.

29. The method of claim 22, wherein the valve includes a petal valve having a plurality of movable petals allowing the first direction of blood flow through the lumen and inhibiting the second direction of blood flow through the lumen.

30. The method of claim 22, wherein the valve includes a funnel valve.

31. The method of claim 22, wherein the valve includes one or more valve seats defining a channel therethrough and a valve body having a head having a plurality of holes, a neck, and a base, the neck being positioned within the channel defined by the one or more valve seats and the head having a greater diameter than the base.

32. The method of claim 22, wherein the sleeve includes a liner bonded to an outer sleeve, the valve being bonded to the liner.

33. The method of claim 32, wherein the stent body is embedded within the sleeve between the liner and the outer sleeve.
